(19) United States
(12) Patent Application Publication
(10) Pub. No.: US 2004/0102442 A1
(43) Pub. Date: May 27, 2004

(54) Remedies for alzheimer's disease

(76) Inventors: Kohji Hanasaki, Osaka-shi (JP); Minoru Ikeda, Osaka-shi (JP); Takashi Ono, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/312,615

(22) PCT Filed: Jun. 27, 2001
(86) PCT No.: PCT/JP01/05482

(30) Foreign Application Priority Data
Jun. 29, 2000 (JP) 2000-195445

(51) Int. Cl. A61K 31/502; A61K 31/498; A61K 31/192

(52) U.S. Cl. 514/248; 514/249; 514/307; 514/569

(57) ABSTRACT

It is provided that type-X sPLA₂ inhibitors are useful in preventing or treating Alzheimer's disease.

### TECHNICAL FIELD

The present invention relates to a composition for the prevention or treatment of Alzheimer's disease which contains an inhibitor against type-X sPLA₂ (secretary PLA₂) as an active ingredient.

### BACKGROUND ART

U.S. Pat. No. 5,478,857 (WO95/17183, JP Laid-Open (Tokuhyo) No. 97/507069) describes that sPLA₂ inhibitors are effective for the treatment of Alzheimer's disease. And it is described in WO99/2403 that type-II sPLA₂ inhibitors are effective for apoptosis associated disease including Alzheimer's disease. However, the above-mentioned documents do not describe that type-X sPLA₂ inhibitors are effective for the treatment of Alzheimer's disease.

### DISCLOSURE OF INVENTION

The inventors of the present invention examined the expression of type-X sPLA₂ in various kinds of human pathological tissues with anti-type-X sPLA₂ antibody. They found the elevated expression of type-X sPLA₂ in some neuronal regions in brain tissues, especially, senile plaques and neurofibrillary tangles regions, from patients of Alzheimer's disease.

The immunohistochemical analysis of each tissue was performed as follows. At first, anti-human type-X sPLA₂ antibody was added to the slides prepared from normal adult cerebral tissues or cerebral tissues prepared from patients of Alzheimer's disease and incubated for several hours. Next, in order to examine the expression of type-X sPLA₂ in the tissues, the expression of type-X sPLA₂ was visualized by using the methods such as the immunohistochemical labeling to detect the type-X sPLA₂ signals. Consequently, the type-X sPLA₂ signals were detected in the slides prepared from cerebral tissues prepared from patients of Alzheimer's disease, suggesting that the expression of type-X sPLA₂ is elevated in cerebral tissues prepared from patients of Alzheimer's disease.

In addition, the inventors of the present invention performed the experiments for neutralization of type-X sPLA₂ signals. Precisely, before the addition of anti-human type-X sPLA₂ antibody to the slides, the slides were incubated with the purified type-X sPLA₂ protein for several hours. Hereafter, the slides were processed as the same procedures as described above to examine the type-X sPLA₂ signals. Consequently, the type-X sPLA₂ signals were disappeared in the slides prepared from cerebral tissues.

Thus, the elevated expression of type-X sPLA₂ was confirmed in cerebral tissues prepared from patients of Alzheimer's disease and the inventors of the present invention achieved the following present invention.

That is to say, the present invention relates to I) a composition for prevention or treatment of Alzheimer's disease which contains a type-X sPLA₂ inhibitor as an active ingredient.

In more detail, the present invention relates to the following II) to XIII).

II) A composition for prevention or treatment of Alzheimer's disease which contains as an active ingredient a compound represented by the formula (I):

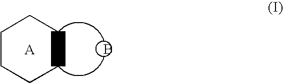

wherein Ring A is represented by the formula (a) to (d):

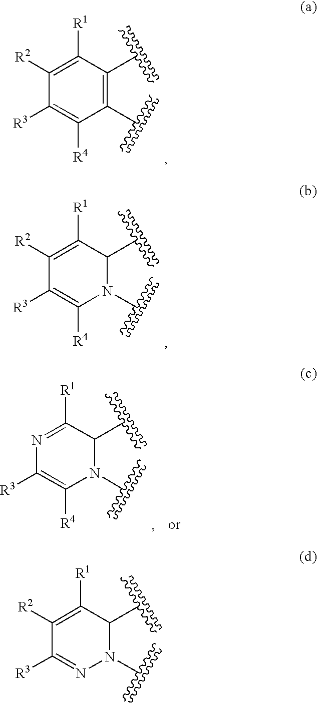

wherein R¹ and R² are each independently hydrogen atom, non-interfering substituent, or —(L¹)-(acidic group) wherein L¹ is an acid linker having an acid linker length of 1 to 5, provided that one of the R¹ and R² is —(L¹)-(acidic group);

R³ and R⁴ are each independently hydrogen atom, non-interfering substituent, carbocyclic group, carbocyclic group substituted with a non-interfering substituent(s), heterocyclic group, or heterocyclic group substituted by a non-interfering substituent(s); and

—B— is represented by the formula (e) to (h):

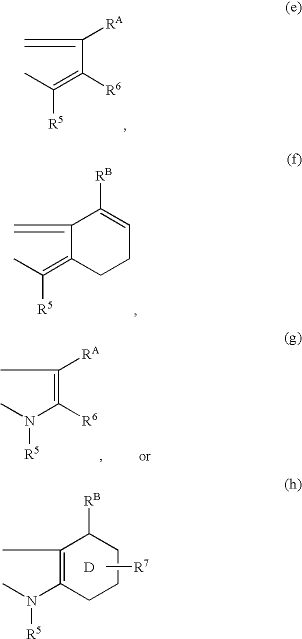

wherein R⁵ is (j) C1 to C20 alkyl, C2 to C20 alkenyl, C2 to C20 alkynyl, carbocyclic group, or heterocyclic group, (k) the group represented by (j) each substituted independently with at least one group selected from non-interfering substituents, or —(L²)—R⁸ wherein L² is a divalent linking group of 1 to 18 atom(s) selected from hydrogen atom(s), nitrogen atom(s), carbon atom(s), oxygen atom(s), and sulfur atom(s), and

R⁸ is a group selected from the groups (j) and (k);

R⁶ is hydrogen atom, halogen, C1 to C3 alkyl, C3 to C4 cycloalkyl, C3 to C4 cycloalkenyl, C1 to C3 alkyloxy, or C1 to C3 alkylthio;

R⁷ is hydrogen atom or non-interfering substituent;

R^{A} is represented by the formula:

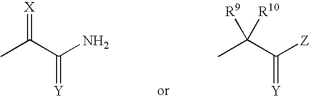

wherein R⁹ and R¹⁰ are each independently hydrogen atom, C1 to C3 alkyl, or halogen;

X and Y are each independently oxygen atom or sulfur atom; and

Z is —NH₂ or —NHNH₂;

R^{B} is —CONH₂ or —CONHNH₂; and,

Ring D is cyclohexene ring or benzene ring;

provided that Ring A is (b), (c), or (d) when —B— is (e) or (f), a prodrug thereof, its pharmaceutically acceptable salt, or its solvate.

III) A composition for prevention or treatment of Alzheimer's disease which contains a compound, a prodrug thereof, its pharmaceutically acceptable salt, or its solvate as described in II) as an active ingredient, wherein R¹ is hydrogen atom or —(L³)—R¹¹ wherein L³ is —OCH₂—, —SCH₂—, —NH—CH₂—, —CH₂—CH₂—, —O—CH(CH₃)—, or —O—CH(CH₂CH₂C₆H₅)—; R¹¹ is —COOH, —CONHSO₂C₆H₅, —SO₃H, or —P(O)(OH)₂; and

R² is hydrogen atom or —(L⁴)—R¹² wherein L⁴ is represented by the formula:

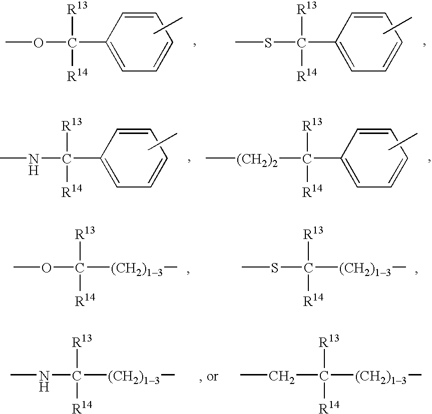

wherein R¹³ and R¹⁴ are each independently hydrogen atom, C1 to C10 alkyl, C1 to C10 aralkyl, carboxy, alkyloxycarbonyl, or halogen; R¹² is —COOH, —SO₃H, or —P(O)(OH)₂, provided R¹ and R² are not hydrogen atom at the same time.

IV) A composition for prevention or treatment of Alzheimer's disease which contains a compound, a prodrug thereof, its pharmaceutically acceptable salt, or its solvate as described in II) or III) as an active ingredient, wherein R³ is hydrogen atom, C1 to C6 alkyl, C3 to C6 cycloalkyl, aryl, or a heterocyclic group and R⁴ is hydrogen atom or halogen.

V) A composition for prevention or treatment of Alzheimer's disease which contains a compound, a prodrug thereof, its pharmaceutically acceptable salt, or its solvate as described in any one of II) to IV) as an active ingredient, wherein R⁵ is —(CH₂)₁₋₆—R¹⁵ wherein R¹⁵ is represented by the formula:

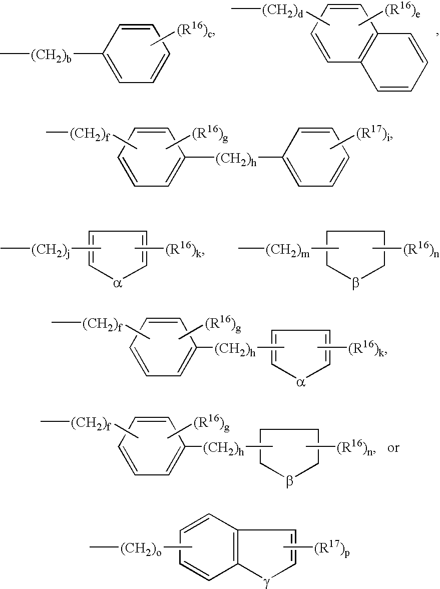

wherein b, d, f, h, j, m, and o are independently an integer from 0 to 2; R¹⁶ and R¹⁷ are each independently halogen, C1 to C10 alkyl, C1 to C10 alkyloxy, C1 to C10 alkylthio, aryloxy, or C1 to C10 haloalkyl; a is oxygen atom or sulfur atom; β is —CH₂— or —(CH₂)₂—; γ is oxygen atom or sulfur atom; c, i, and p are independently an integer from 0 to 5; e is an integer from 0 to 7; g is an integer from 0 to 4; k and n are each independently an integer from 0 to 3.

VI) A composition for prevention or treatment of Alzheimer's disease which contains a compound, a prodrug thereof, its pharmaceutically acceptable salt, or its solvate as described in V) as an active ingredient, wherein R⁵ is —CH₂—R¹⁸ wherein R¹⁸ is represented by the formula:

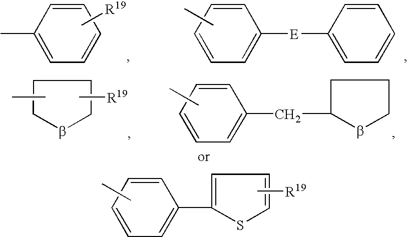

wherein β is —CH₂— or —(CH₂)₂—; R¹⁹ is hydrogen atom, C1 to C3 alkyl, or halogen; E is a bond, —CH₂— or —O—.

VII) A composition for prevention or treatment of Alzheimer's disease which contains a compound, a prodrug thereof, its pharmaceutically acceptable salt, or its solvate as described in any one of II) to VI) as an active ingredient, wherein R¹ is —OCH₂COOH.

VIII) A composition for prevention or treatment of Alzheimer's disease which contains a compound, a prodrug thereof, its pharmaceutically acceptable salt, or its solvate as described in any one of II) to VII) as an active ingredient, wherein R² is hydrogen atom.

IX) A composition for prevention or treatment of Alzheimer's disease which contains a compound, a prodrug thereof, its pharmaceutically acceptable salt, or its solvate as described in any one of II) to VIII) as an active ingredient, wherein R⁶ is C1 to C3 alkyl.

X) A composition for prevention or treatment of Alzheimer's disease which contains a compound, a prodrug thereof, its pharmaceutically acceptable salt, or its solvate as described in any one of II) to IX)as an active ingredient, wherein R^{A} is —CH₂CONH₂ or —COCONH₂.

XI) A composition for prevention or treatment of Alzheimer's disease which contains a compound as an active ingredient represented by the formula:

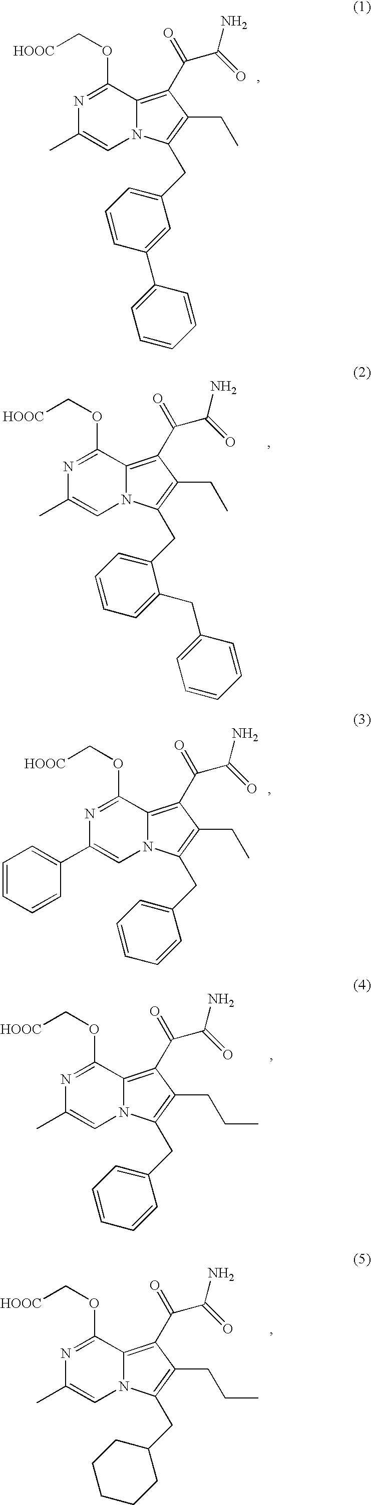

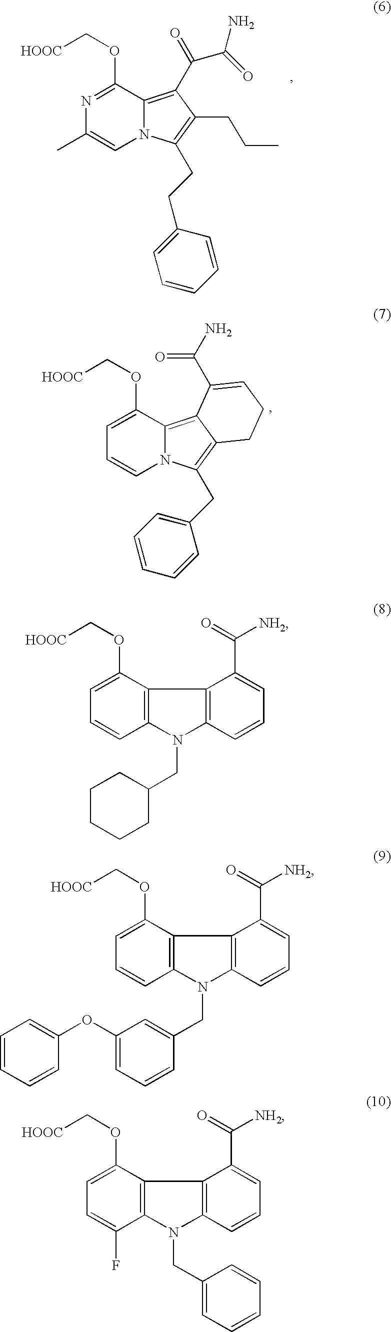

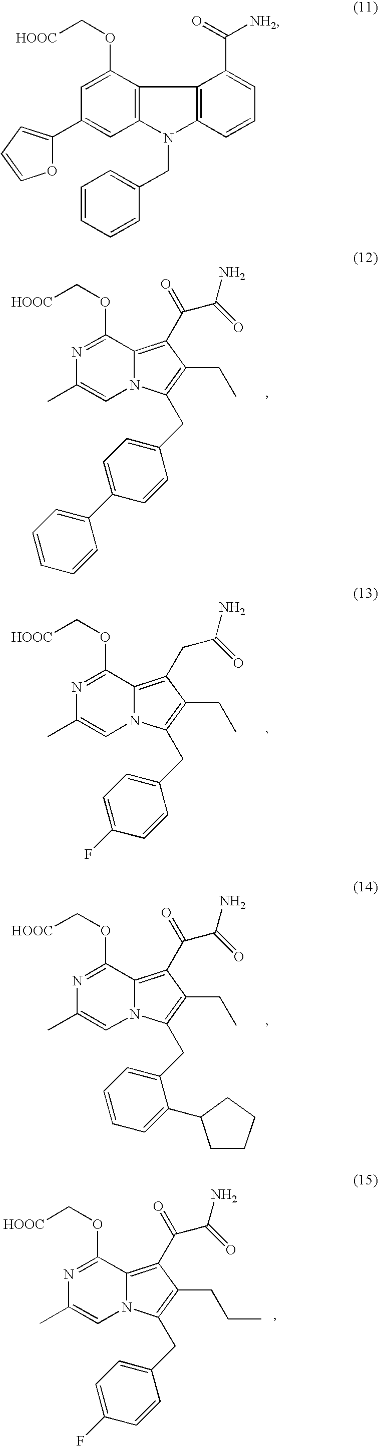

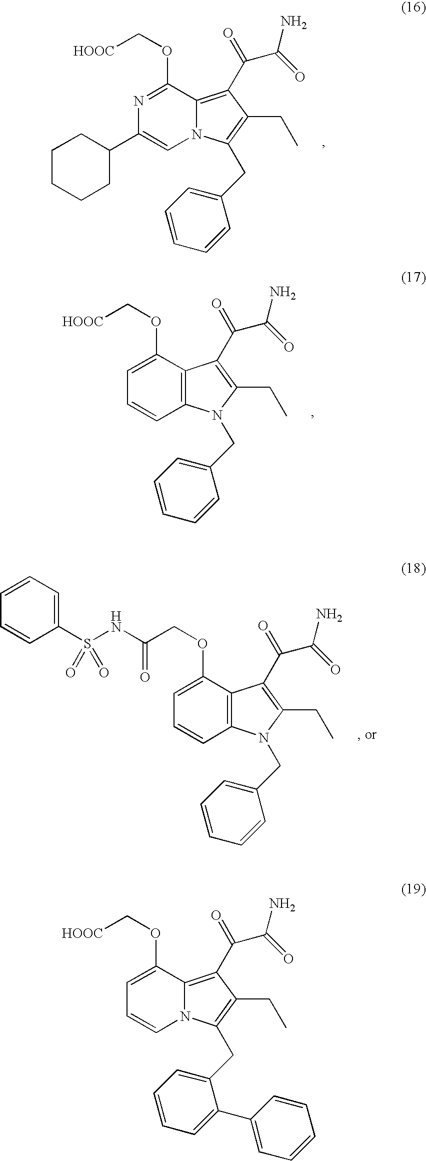

a prodrug thereof, its pharmaceutically acceptable salt, or its solvate.

XII) Use of a type-X sPLA₂ inhibitor for the preparation of a medicament for the treatment of Alzheimer's disease.

XIII) Use as described in XII) wherein the type-X sPLA₂ inhibitor is the compound described in any one of II) to XI).

XIV) A method of treating a mammal, including a human, to alleviate the pathological effects of Alzheimer's disease, which comprises administration to said mammal of a type-X sPLA₂ inhibitor in a pharmaceutically effective amount.

XV) A method as described in XIV) wherein the type-X sPLA₂ inhibitor is the compound described in any one of II) to XI).

The present invention is illustrated in detail as follows

Type-X sPLA₂ inhibitors mean compounds which have an inhibitory activity against type-X sPLA₂ and other inhibitory activities such as inhibitory activities against other enzymes or affinities for any receptors. Namely, the inhibitors include any compound having stronger activities against type-X sPLA₂ than that having no such activities in the evaluation test therefore. Especially, type-X sPLA₂ selective inhibitors are preferred as type-X sPLA₂ inhibitors of the present invention. For example, compounds whose IC₅₀ values against type-X sPLA₂ are 1 μM or less in the experiment of Example 2 are preferred. Compounds having IC₅₀ values 100 nM or less are more preferred.

A compound having type-X sPLA₂ inhibitory activities, having one or more of chiral center(s), may exist as an optically active member. Likewise, a compound containing alkenyl or alkenylene, may be a cis- or trans-isomer. Mixtures of R- and S-isomers as well as of cis- and trans-isomers, and mixtures of R- and S-isomers containing a racemic mixture are included in the scope of the present invention. An asymmetric carbon atom may exist also in a substituent such as alkyl group. All such isomers and mixtures are included in the present invention. A specified stereoisomer can be manufactured by subjecting to stereospecific reaction well known to those skilled in the art applying a previously separated starting material having an asymmetrical center or by preparing a mixture of stereoisomers and separating the mixture in accordance with a well-known manner.

Prodrug is a derivative of a compound with type-X sPLA₂ inhibitory activities, having a group which can be decomposed chemically or metabolically, and becoming pharmaceutically active by solvolysis or in vivo under a physiological condition. Although the derivative, acid derivative or basic derivative, exhibits activity, an acid derivative is more advantageous in solubility, tissue affinity, and release control in mammal organism (Bungard, H., Design of Prodrugs, pp. 7-9, 21-24, Elsevier, Amsterdam, 1985). For instance, prodrugs, including an acid derivative such as an ester which is prepared by reacting a basal acid compound with a suitable alcohol, or an amide which is prepared by reacting a basal acid compound with a suitable amine, are well known to those skilled in the art. Simple aliphatic or aromatic esters derived from acid groups contained in the compounds according to the present invention are preferable prodrugs. Particularly preferred esters as prodrugs are C1-C6 alkylester (e.g. methyl ester, ethyl ester). Double ester such as (acyloxy)alkyl ester or ((alkyloxycarbonyl)oxy)-alkyl ester type prodrugs may be optionally manufactured.

When a compound having type-X sPLA₂ inhibitory activities has an acidic or basic functional group, a variety of salts having a higher water solubility and more physiologically suitable properties than those of the original compound can be formed. An example of typical pharmaceutically acceptable salts includes salts with alkali metal and alkaline earth metal such as lithium, sodium, potassium, magnesium, aluminum and the like, but it is to be noted that such pharmaceutically acceptable salts are not limited thereto. A salt is easily manufactured from a free acid by either treating an acid in a solution with a base, or allowing an acid to be in contact with an ion exchange resin. Addition salts of the compounds having type-X sPLA₂ inhibitory activities with relatively non-toxic inorganic bases and organic bases, for example, amine cation, ammonium, and quaternary ammonium derived from nitrogenous bases having a basicity sufficient for forming a salt of the compounds of the present invention are included in the definition of “pharmaceutically acceptable salts”. (e.g., S. M. Berge et al., “Pharmaceutical Salts,” J. Phar. Sci., 66, 1-19 (1977)). Furthermore, basic groups of a compound having type-X sPLA₂ inhibitory activities are reacted with a suitable organic or inorganic acid to form salts such as acetates, benzenesulfonates, benzoates, bicarbonates, bisulfates, bitartrates, borates, bromides, camsylates, carbonates, chlorides, clavulanates, citrates, edetates, edisylates, estolates, esylates, fluorides, fumarates, gluceptates, gluconates, glutamates, glycolylarsanilates, hexylresorcinates, hydroxynaphthoates, iodides, isothionates, lactates, lactobionates, laurates, malates, maleates, mandelates, mesylates, methylbromides, methylnitrates, methylsulfates, mucates, napsylates, nitrates, oleates, oxalates, palmitates, pantothenates, phosphates, polygalacturonates, salicylates, stearates, subacetates, succinates, tannates, tartrates, tosylates, trifluoroacetates, trifluoromethanesulfonates, valerates and the like.

The solvate includes solvates with organic solvents and/or hydrates. In case of forming a hydrate, a questioned compound may be coordinated with a suitable number of water molecules.

The term “pharmaceutically acceptable” means that carriers, diluents, or additives are compatible with other ingredients in a formulation and are not harmful for recipients.

“Alzheimer's disease” is a progressive derangement assumed disturbance of memory or disturbance of orientation, comprises dwarf of cerebrum, especially temporal lobe, on pathology, and change of neurofibril and senile plaque on histology. Based on the experiments, the inventors of the present invention confirmed the elevated expression of type-X sPLA₂ in some neurons in brain tissues from patients of Alzheimer's disease, especially in senile plaques and neurofibrillary tangles. In particular, the compounds in this invention are useful for prevention or treatment of Alzheimer's disease.

In the present specification, the term “alkyl” employed alone or in combination with other terms means a straight- or branched chain monovalent hydrocarbon group having a specified number of carbon atoms. An example of the alkyl includes methyl, ethyl, n-propyl, isopropyl, n-butyl, isobutyl, sec-butyl, tert-butyl, n-pentyl, n-hexyl, n-heptyl, n-octyl, n-nonyl, n-decanyl, n-undecanyl, n-dodecanyl, n-tridecanyl, n-tetradecanyl, n-pentadecanyl, n-hexadecanyl, n-heptadecanyl, n-octadecanyl, n-nonadecanyl, n-eicosanyl and the like.

The term “alkenyl” employed alone or in combination with other terms in the present specification means a straight- or branched chain monovalent hydrocarbon group having a specified number of carbon atoms and at least one double bond. An example of the alkenyl includes vinyl, allyl, propenyl, crotonyl, isopentenyl, a variety of butenyl isomers and the like.

The term “alkynyl” used in the present specification means a straight or branched chain monovalent hydrocarbon group having a specified number of carbon atoms and at least one triple bond. The alkynyl may contain (a) double bond(s). An example of the alkynyl includes ethynyl, propynyl, 6-heptynyl, 7-octynyl, 8-nonynyl and the like.

The term “carbocyclic group” used in the present specification means a group derived from a saturated or unsaturated, substituted or unsubstituted 5 to 14 membered, preferably 5 to 10 membered, and more preferably 5 to 7 membered organic nucleus whose ring forming atoms (other than hydrogen atoms) are solely carbon atoms. A group containing two to three of the carbocyclic group is also included in the above stated group. An example of typical carbocyclic groups includes cycloalkyl such as cyclopropyl, cyclobutyl, cyclopentyl, cyclohexyl, cycloheptyl, and cyclooctyl, cycloalkenyl such as cyclobutylenyl, cyclopentenyl, cyclohexenyl, cycloheptenyl, and cyclooctenyl, phenyl, naphthyl, norbornyl, bicycloheptadienyl, indenyl, stilbenyl, terphenylyl, phenylcyclohexenyl, acenaphthyl, anthryl, biphenylyl, bibenzyl, and a phenylalkylphenyl derivative represented by the formula (II):

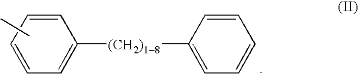

Phenyl, cycloalkyl or the like is preferred as a carbocyclic groups in the R³ and R⁴.

The term “heterocyclic group” used in the present specification means a group derived from monocyclic or polycyclic, saturated or unsaturated, substituted or unsubstituted heterocyclic nucleus having 5 to 14 ring atoms and containing 1 to 3 hetero atoms selected from the group consisting of nitrogen atom, oxygen atom, and sulfur atom. An example of the heterocyclic group includes pyridyl, pyrrolyl, furyl, benzofuryl, thienyl, benzothienyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, dibenzothiophenyl, indazolyl, imidazo[1,2-a]pyridinyl, benzotriazolyl, anthranilyl, 1,2-benzisoxazolyl, benzoxazolyl, benzothiazolyl, purinyl, puridinyl, dipyridinyl, phenylpyridinyl, benzylpyridinyl, pyrimidinyl, phenylpyrimidinyl, pyrazinyl, 1,3,5-triazinyl, quinolyl, phthalazinyl, quinazolinyl, quinoxalinyl, and the like.

Furyl, thienyl or the like is preferred as a heterocyclic group in the R³ and R⁴.

Preferred carbocyclic and heterocyclic groups in R⁵ represented by the formula:

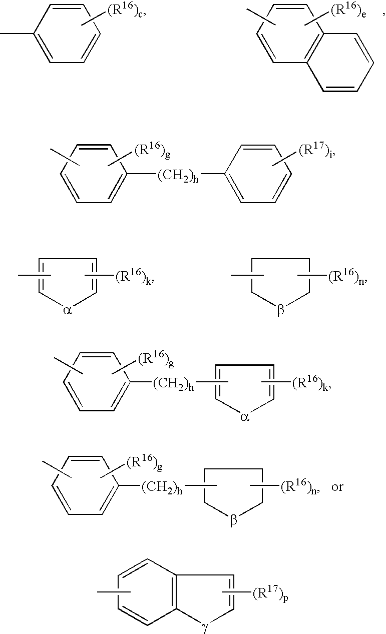

wherein h is an integer from 0 to 2, R¹⁶ and R¹⁷ are each independently halogen, C1-C10 alkyl, C1-C10 alkyloxy, C1-C10 alkylthio, aryloxy, or C1-C10 haloalkyl, α is oxygen atom or sulfur atom, β is —CH₂— or —(CH₂)₂—, γ is oxygen atom or sulfur atom, c, i, and p are each independently an integer from 0 to 5, e is an integer from 0 to 7, g is an integer from 0 to 4, k and n are each independently an integer from 0 to 3. When the above c, e, g, i, k, n, and/or p are 2 or more, a plural number of R¹⁶ or R¹⁷ may be different from one another. When R¹⁶ is a substituent on the naphthyl group, the substituent may be substituted at any arbitrary position on the naphthyl group.

A more preferable example includes a group represented by the formula:

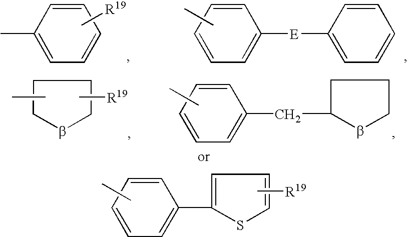

wherein R¹⁹ is hydrogen atom, C1-C3 alkyl or halogen; E is a bond, —CH₂—, or —O—; β is —CH₂— or —(CH₂)₂— as defined above.

The above-mentioned “carbocyclic ring” C1-C3 alkyl and the above-mentioned “heterocyclic ring” C1-C3 alkyl, or the like is preferred as a group in the R⁵.

The term “non-interfering substituent” in the present specification means a group suitable for substitution of the above mentioned “carbocyclic group”, “heterocyclic group”, and basic skeleton. An example of the non-interfering substituents includes C1-C10 alkyl, C2-C6 alkenyl, C2-C6 alkynyl, C7-C12 aralkyl such as benzyl and phenethyl, C7-C12 alkaryl, C3-C8 cycloalkyl, C3-C8 cycloalkenyl, phenyl, tolyl, xylyl, biphenylyl, C1-C10 alkyloxy, C1-C6 alkyloxy C1-C6 alkyl such as methyloxymethyl, ethyloxymethyl, methyloxyethyl, and ethyloxyethyl, C1-C6 alkyloxy C1-C6 alkyloxy such as methyloxymethyloxy and methyloxyethyloxy, C1-C6 alkylcarbonyl such as methylcarbonyl and ethylcarbonyl, C1-C6 alkylcarbonylamino such as methylcarbonylamino and ethylcarbonylamino, C1-C6 alkyloxyamino such as methyloxyamino and ethyloxyamino, C1-C6 alkyloxyaminocarbonyl such as methyloxyaminocarbonyl and ethyloxyaminocarbonyl, mono or di C1-C6 alkylamino such as methylamino, ethylamino, dimethylamino, and ethylmethylamino, C1-C10 alkylthio, C1-C6 alkylthiocarbonyl such as methylthiocarbonyl and ethylthiocarbonyl, C1-C6 alkylsulfinyl such as methylsulfinyl and ethylsulfinyl, C1-C6 alkylsulfonyl such as methylsulfonyl and ethylsulfonyl, C2-C6 haloalkyloxy such as 2-chloroethyloxy and 2-bromoethyloxy, C1-C6 haloalkylsulfonyl such as chloromethylsulfonyl and bromomethylsulfonyl, C1-C10 haloalkyl, C1-C6 hydroxyalkyl such as hydroxymethyl and hydroxyethyl, C1-C6 alkyloxycarbonyl such as methyloxycarbonyl and ethyloxycarbonyl, —(CH₂)₁₋₈—O—(C1-C6 alkyl), benzyloxy, aryloxy such as phenyloxy, arylthio such as phenylthio, —(CONHSO₂R²⁰) wherein R²⁰ is C1-C6 alkyl or aryl, —CHO, amino, amidino, halogen, carbamyl, carboxyl, carbalkoxy, —(CH₂)₁₋₈—COOH such as carboxymethyl, carboxyethyl, and carboxypropyl, cyano, cyanoguanidino, guanidino, hydrazide, hydrazino, hydroxy, hydroxyamino, nitro, phosphono, —SO₃H, thioacetal, thiocarbonyl, C1-C6 carbonyl, a carbocyclic group, a heterocyclic group and the like. These are optionally substituted with one or more substituents selected from the group consisting of C1-C6 alkyl, C1-C6 alkyloxy, C2-C6 haloalkyloxy, C1-C6 haloalkyl, and halogen.

Preferable are halogen, C1-C6 alkyl, C1-C6 alkyloxy, C1-C6 alkylthio, and C1-C6 haloalkyl as the “non-interfering substituent” of “substituted with non-interfering substituent” in the R³, R⁴, and R⁵. More preferable are halogen, C1-C3 alkyl, C1-C3 alkyloxy, C1-C3 alkylthio, and C1-C3 haloalkyl.

Preferable are C1-C6 alkyl, aralkyl, C1-C6 alkyloxy, C1-C6 alkylthio, C1-C6 hydroxyalkyl, C2-C6 haloalkyloxy, halogen, carboxy, C1-C6 alkyloxycarbonyl, aryloxy, arylthio, a carbocyclic group, and a heterocyclic group as the “non-interfering substituent” in the R¹, R², R³, R⁴, and R⁷. More preferable are C1-C6 alkyl, aralkyl, carboxy, C1-C6 hydroxyalkyl, phenyl, and C1-C6 alkyloxycarbonyl.

The term “halogen” in the present specification means fluorine, chlorine, bromine, and iodine.

The term “cycloalkyl” in the present specification means a monovalent cyclic hydrocarbon group having a specified number of carbon atoms. An example of the cycloalkyl includes cyclopropyl, cyclobutyl, cyclopentyl, cyclohexyl, cycloheptyl, cyclooctyl and the like.

The term “cycloalkenyl” in the present specification means a monovalent cyclic hydrocarbon group having a specified number of carbon atoms and at least one double bond(s). An example of the cycloalkenyl includes 1-cyclopropenyl, 2-cyclopropenyl, 1-cyclobutenyl, 2-cyclobutenyl and the like.

In the present specification, an example of “alkyloxy” includes methyloxy, ethyloxy, n-propyloxy, isopropyloxy, n-butyloxy, n-pentyloxy, n-hexyloxy and the like.

In the present specification, an example of “alkylthio” includes methylthio, ethylthio, n-propylthio, isopropylthio, n-butylthio, n-pentylthio, n-hexylthio and the like.

The term “acidic group” in the present specification means an organic group functioning as a proton donor capable of hydrogen bonding when attached to a basic skeleton through a suitable linking atom (hereinafter defined as “acid linker”). An example of the acidic group includes a group represented by the formula:

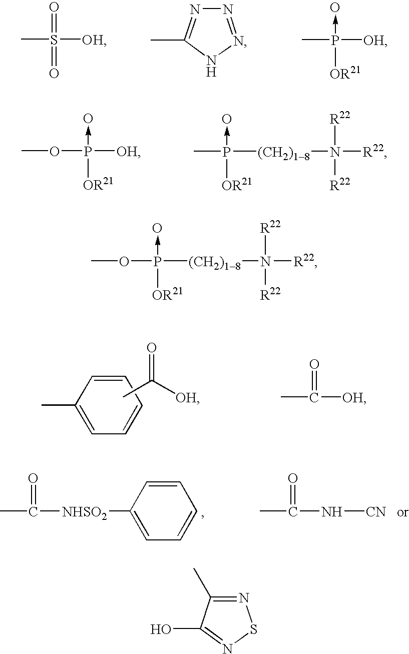

wherein R²¹ is hydrogen atom, a metal, or C1-C10 alkyl; each R²² is independently hydrogen atom or C1-C10 alkyl, provided that at least one of R²¹ or R²² is hydrogen atom in case of an acidic group having both R²¹ and R²². Preferable is —COOH, —SO₃H, —CONHSO₂C₆H₅, or P(O)(OH)₂. More preferable is —COOH.

The term “acid linker” in the present specification means a divalent linking group represented by a symbol —(L¹)—, and it functions to join a basic skeleton to an “acidic group” in the general relationship. An example of it includes a group represented by the formula:

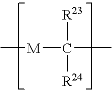

wherein M is —CH₂—, —O—, —N(R²⁵)—, or —S— wherein R²³ and R²⁴ are each independently hydrogen atom, C1-C10 alkyl, aryl, aralkyl, carboxy, or halogens and a group represented by the formula:

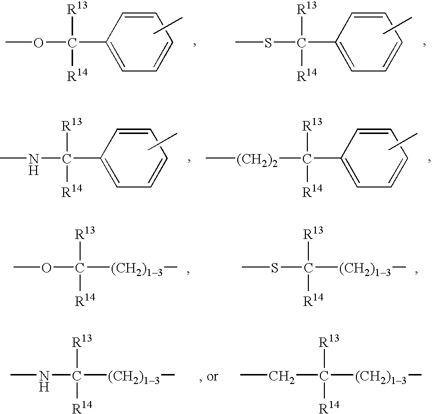

wherein R¹³ and R¹⁴ are each independently hydrogen atom, C1-C10 alkyl, C1-C10 aralkyl, carboxy, alkyloxycarbonyl, or halogen. Preferable are —O—CH₂—, —S—CH₂—, —N(R²⁵)—CH₂—, —CH₂—CH₂—, —O—CH(CH₃)—, or —O—CH((CH₂)₂C₆H₅)— wherein R²⁵ is C1-C6 alkyl. More preferable is —O—CH₂— or —S—CH₂—.

In the present specification, the term “acid linker length” means the number of atoms (except for hydrogen atoms) in the shortest chain of a linking group —(L¹)— which connects a basic skeleton with the “acidic group”. The presence of a carbocyclic ring in —(L¹)— counts as the number of atoms approximately equivalent to the calculated diameter of the carbocyclic ring. Thus, a benzene and cyclohexane ring in the acid linker counts as two atoms in calculating the length of —(L¹)—. A preferable length is 2 to 3.

The term “haloalkyl” in the present specification means the aforementioned “alkyl” substituted with the aforementioned “halogen” at arbitrary position(s). An example of the haloalkyl includes chloromethyl, trifluoromethyl, 2-chloromethyl, 2-bromomethyl and the like.

The term “hydroxyalkyl” in the present specification means the aforementioned “alkyl” substituted with hydroxy at arbitrary position(s). An example of the hydroxyalkyl includes hydroxymethyl, 2-hydroxyethyl, 3-hydroxypropyl and the like. In this case, hydroxymethyl is preferable.

In the present specification, the term “haloalkyl” in “haloalkyloxy” is the same as defined above. An example of it includes 2-chloroethyloxy, 2-trifluoroethyloxy, 2-chloroethyloxy and the like.

The term “aryl” in the present specification means a monocyclic or condensed cyclic aromatic hydrocarbon. An example of the aryl includes phenyl, 1-naphthyl, 2-naphthyl, anthryl and the like. Particularly, phenyl and 1-naphthyl are preferred.

The term “aralkyl” in the present specification means a group wherein the aforementioned “alkyl” is substituted with the above-mentioned “aryl”. Such aryl may have a bond at any substitutable position. An example of it includes benzyl, phenethyl, phenylpropyl such as 3-phenylpropyl, naphthylmethyl such as 1-naphthylmethyl and the like.

An example of the “alkyloxycarbonyl” in the present specification includes methyloxycarbonyl, ethyloxycarbonyl, n-propyloxycarbonyl and the like.

An example of the “aryloxy” in the present specification includes phenyloxy and the like.

An example of the “arylthio” in the present specification includes phenylthio and the like.

The term “halophenyl” in the present specification means phenyl substituted with the aforementioned “halogen” at one or more position(s). An example of the halophenyl includes fluorophenyl, chlorophenyl, bromophenyl, iodophenyl, difluorophenyl, dichlorophenyl, dibromophenyl, trifluorophenyl, trichlorophenyl, tribromophenyl, chlorofluorophenyl, bromochlorophenyl, and the like.

The term “cyclohexene ring” of D ring in the present specification means a cyclohexene ring having only one double bond at the condensation part with the adjacent ring.

Preferable combinations of “A ring” and “—B—” are represented by the following (m)-(r):

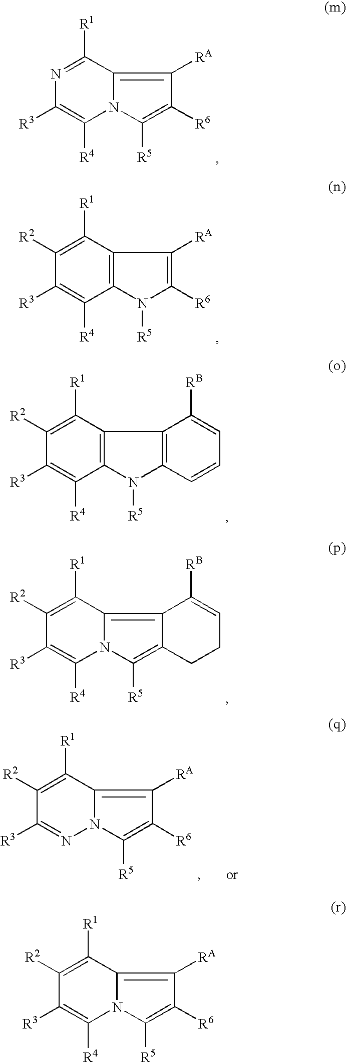

Particularly, combinations represented by (m)-(p) are preferred.

Furthermore, compounds represented by formula (1) to (19) are most preferred.

### BEST MODE FOR CARRYING OUT THE INVENTION

The present invention relates to the prevention or treatment of Alzheimer's disease by a type-X sPLA₂ inhibitor. The type-X sPLA₂ inhibitor may be known one and selected from sPLA2 inhibitors, for example, compounds described in EP-620214 (JP Laid-Open (Tokukai) No. 95/010838, U.S. Pat. No. 5,578,634), EP-620215 (JP Laid-Open (Tokukai) No. 95/025850, U.S. Pat. No. 5,684,034), EP-675110 (JP Laid-Open (Tokukai) No. 95/285933, U.S. Pat. No. 5,654,326), WO 96/03120 (JP Laid-Open No. 98/505336), WO 96/03376 (JP Laid-Open No. 98/503208, U.S. Pat. No. 5,641,800), WO 96/03383 (JP Laid-Open No. 98/505584), WO 97/21664 (EP-779271), WO 97/21716 (EP-779273), WO 98/18464 (EP839806), WO98/24437 (EP846687), WO98/24756, WO98/24794, WO98/25609, WO99/51605, WO99/59999 and the like, or parabromophenacyl-bromide, mepacrine, manoalide, thielocin A₁ and the like.

As another type-X sPLA₂ inhibitor, can be used the compounds represented in PCT/JP00/07024 by the formula:

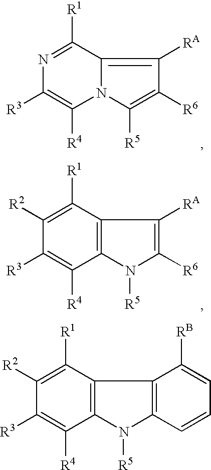

wherein R¹, R², R³, and R⁴ are each independently hydrogen atom, a non-interfering substituent(s) and the like, R⁵ is carbocyclic groups, heterocyclic groups, R⁶ is hydrogen atom, C1-C3alkyl and the like, R^{A} is —COCONH₂ and the like, R^{B} is —CONH₂, and the like.

Further, compounds identified as type-X sPLA₂ inhibitors by the following procedure and the like may be used in the present invention.

At first, a cell expressing human type-X sPLA₂ is prepared. That is, cDNA sequence encoding human type-X sPLA₂ (Cupillard et al., J. Biol. Chem, 1997, 272, 15745-15752) is inserted into an expression vector for mammalian cells. The obtained expression vector is transfected into the host cell and the cell stably expressing human type-X sPLA₂ is obtained.

Next, the above-mentioned transfected cell is cultured in medium and its culture supernatant is used for the measurement of each enzyme activity. In order to identify and evaluate an inhibitor of type-X sPLA₂, the following chromogenic assay is utilized. A general explanation for this assay is described in “Analysis of Human Synovial Fluid Phospholipase A₂ on Short Chain Phosphatidylcholine-Mixed Micelles: Development of a Spectrophotometric Assay Suitable for a Micortiterplate Reader” (Analytical Biochemistry, 204, pp 190-197, 1992 by Laure. J. Reynolds. Lori L. Hughes and Edward A. Dennis.

Several kinds of the compounds represented by the formula (I) can be synthesized in accordance with the methods described in PCT/JP00/07024, EP-620214 (JP Laid-Open (Tokukai) No. 95/010838, U.S. Pat. No. 5,578,634), EP-620215 (JP Laid-Open (Tokukai) No. 95/025850, U.S. Pat. No. 5,684,034), EP-675110 (JP Laid-Open (Tokukai) No. 95/285933, U.S. Pat. No. 5,654,326), WO 96/03120 (JP Laid-Open No. 98/505336), WO 96/03383 (JP Laid-Open No. 98/505584), WO 98/18464 (EP839806), WO99/51605, WO99/59999 and the like.

The composition for treatment or prevention of Alzheimer's disease in the present invention may be administered to a patient through a variety of routes including oral, aerosol, rectal, percutaneous, subcutaneous, intravenous, intramuscular, and nasal routes. A formulation according to the present invention may be manufactured by combining (for example, admixing) a curatively effective amount of a compound of the present invention with a pharmaceutically acceptable carrier or diluent. The formulation of the present invention may be manufactured with the use of well-known and easily available ingredients in accordance with a known method.

In case of manufacturing a composition of the present invention, active ingredients are admixed, or diluted with a carrier, or they are contained in a carrier in the form of capsule, sacheier, paper, or another container. In case of functioning a carrier as a diluent, the carrier is a solid, semi-solid, or liquid material which functions as a medium. Accordingly, a formulation according to the present invention may be produced in the form of tablet, pill, powder medicine, intraoral medicine, elixir agent, suspending agent, emulsifier, dissolving agent, syrup agent, aerosol agent (solid in liquid medium), and ointment. Such a formulation may contain up to 10% of an active compound. It is preferred to formulate a compound having activities for the treatment or prevention of Alzheimer's disease prior to administration.

Any suitable carrier well known by those skilled to the art may be used for the formulation. In such formulation, a carrier is in the form of solid, liquid, or a mixture thereof. For instance, a compound having type-X sPLA₂ inhibitory activities is dissolved into 4% dextrose/0.5% sodium citrate aqueous solution so as to be 2 mg/mL concentration for intravenous injection. Solid formulation includes powder, tablet, and capsule. Solid carrier consists of one or more of material(s) for serving also as fragrant, lubricant, dissolving agent, suspension, binder, tablet disintegrator, capsule. A tablet for oral administration contains a suitable excipient such as calcium carbonate, sodium carbonate, lactose, calcium phosphate and the like together with a disintegrator such as corn starch, alginic acid and the like and/or a binder such as gelatin, acacia and the like, and a lubricant such as magnesium stearate, stearic acid, talc and the like.

In a powder medicine, a carrier is a finely pulverized solid which is blended with finely pulverized active ingredients. In a tablet, active ingredients are admixed with a carrier having required binding power in a suitable ratio, and it is solidified in a desired shape and size. Powder medicine and tablet contain about 1 to about 99% by weight of the active ingredients being novel compounds according to the present invention. An example of suitable solid carriers includes magnesium carbonate, magnesium stearate, talc, sugar, lactose, pectin, dextrin, starch, gelatin, tragacanth gum, methyl cellulose, sodium carboxymethylcellulose, low-melting wax, and cocoa butter.

An axenic liquid formulation contains suspending agent, emulsifier, syrup agent, and elixir agent. Active ingredients may be dissolved or suspended into a pharmaceutically acceptable carrier such as sterile water, a sterile organic solvent, a mixture thereof and the like. Active ingredients may be dissolved frequently into a suitable organic solvent such as propylene glycol aqueous solution. When finely pulverized active ingredients are dispersed into aqueous starch, sodium carboxymethylcellulose solution, or suitable oil, the other compositions can be prepared.

The dosage varies with the conditions of the disease, administration route, age and body weight of patient. In the case of intravenous administration, the dosage can generally be between 0.01 to 10 mg/kg/h for adult, preferably 0.1 to 1 mg/kg/h.

### EXAMPLE

### Example 1

### Preparation of Cells Expressing Human Type-X sPLA₂ and their Culture Supernatants

cDNA sequence encoding human type-X sPLA₂ (Cupillard et al., J. Biol. Chem, 1997, 272, 15745-15752) was inserted into the downstream region of the promoter of pSVL SV40 Late Promoter Expression Vector (Amersham Pharmacia Biotech Inc.) that is an expression vector for mammalian cells. The obtained expression vector was transfected into the host CHO cells with a LipofectAMINE reagent (Gibco BRL Inc.) according to the manufacture's instruction to obtain the CHO cells stably expressing human type-X sPLA₂. The transfected cell was cultured in A-MEM medium containing 10% fetal calf serum for 3 days and its culture supernatant was used for the measurement of each enzyme activity.

### Example 2

### Inhibition Test

In order to identify and evaluate an inhibitor of type-X sPLA₂, the following chromogenic assay is utilized. This assay has been applied for high volume screening using a 96-well microtiterplate. A general explanation for this assay is described in “Analysis of Human Synovial Fluid Phospholipase A₂ on Short Chain Phosphatidylcholine-Mixed Micelles: Development of a Spectrophotometric Assay Suitable for a Micortiterplate Reader” (Analytical Biochemistry, 204, pp 190-197, 1992 by Laure. J. Reynolds. Lori L. Hughes and Edward A. Dennis.

Test compounds (or solvent blank) were added according to the alignment of plates that had been previously set. Human type-X sPLA₂ was incubated (30 min at 40° C. (15 μl/well)) with diheptanoylthio PC (1 mM) in the presence of Triton X-100 (0.3 mM) and 5,5′-dithiobis(2-nitrobenzoic acid) (125 μM) in Tris-HCl buffer (25 mM, pH 7.5) containing CaCl₂ (10 mM), KCl (100 mM), and bovine serum albumin (1.0 mg/mL). The changes in the absorbance at 405 nm were measured and the inhibition activities were calculated.

The IC₅₀ value was determined by plotting the log concentration of the above-mentioned compounds (1)-(19) with respect to their inhibition values within 10% to 90% inhibitory range.

Results of the type-X sPLA₂ inhibition test is shown in the following Table 1.

TABLE 1

| Compound | | Compound | |
|---|---|---|---|
| No. | IC₅₀(nM) | No. | IC₅₀(nM) |
| 1 | 10 | 11 | 10 |
| 2 | 10 | 12 | 16 |
| 3 | 5 | 13 | 19 |
| 4 | 27 | 14 | 9 |
| 5 | 12 | 15 | 17 |
| 6 | 17 | 16 | 7 |
| 7 | 5 | 17 | 12 |
| 8 | 3 | 18 | 16 |
| 9 | 13 | 19 | 26 |
| 10 | 12 | | |

### Example 3

### Immunohistochemical Analysis in Human Cerebrum of Brain Tissues with Anti-Type-X sPLA₂ Antibody

In this experiment, anti-type-X sPLA₂ antibody which was described in “The Journal of Biological Chemistry Vol. 274, No. 48, pp.34203-34211 1999” was used. Paraffin embedded preparations of human Alzheimer's disease brain cerebrum tissues and corresponding normal tissues were purchased from Biochain Inc. (San Leandro, Calif.). The tissue sections in the slides were dewaxed, treated in methanol containing 0.3% H₂O₂ for 30 min to remove the endogenous peroxidase activity and incubated with 5% normal goat serum for 20 min. The slides were then incubated with anti-type-X sPLA₂ antibody (6 μg/mL) in PBS containing 0.1% bovine serum albumin for 14 hr at 4° C. After washing with PBS, they were incubated with biotin-conjugated goat anti-rabbit IgG antibody for 30 min followed by treatment with peroxidase labelled avidin-biotin complex reagent (Vector Laboratories). After washing, the samples were processed with 200 μg/ml diaminobenzidine hydrochloride substrate dissolved in 50 mmol/L Tris-HCl (pH 7.6) containing 0.006% H₂O₂ for 10 min resulting in the appearance of color dependent on the peroxidase activity to visualize the type-X sPLA₂ expression in the tissue preparations. In addition, the nuclei were counterstained with Gill's 0.4% hematoxylin solution. Positive signals representative for type-X sPLA₂ expression was visualized as a dark-brownish color of diaminobenzidine deposit. The neutralization of type-X sPLA₂ specific signals was conducted by incubating anti-type-X sPLA₂ antibody with purified type-X sPLA₂ protein (60 μg/ml) for 2 hr before the addition to the slides.

Consequently, positive signals representative for type-X sPLA₂ expression were not obviously detected in normal brain cerebrum tissues, but strongly detected in some neuronal regions, especially in senile plaque and neurofibrillary tangle regions, in the cerebrum tissues prepared from patients of Alzheimer's disease. Since the addition of type-X sPLA₂ protein resulted in abolishment of the signals, they were verified as the specific signals for type-X sPLA₂. In addition, there was no positive signal when IgG prepared from non-immunized rabbit was used. Taken together, these findings suggest that the expression of type-X sPLA₂ protein was greatly elevated in human Alzheimer's disease brain tissues.

### Formulation Example

It is to be noted that the following Formulation Examples 1 to 8 are mere illustration, but not intended to limit the scope of the invention. The term “active ingredient” means the compounds having an effect for prevention or treatment of Alzheimer's disease, the prodrugs thereof, their pharmaceutical acceptable salts, or their hydrate.

### Formulation Example 1

Hard gelatin capsules are prepared using of the following ingredients:

| | Dose | |
|---|---|---|
| | (mg/capsule) | |
| Active ingredient | 250 | |
| Starch, dried | 200 | |
| Magnesium stearate | 10 | |
| Total | 460 | mg |

### Formulation Example 2

A tablet is prepared using of the following ingredients:

| | Dose | |
|---|---|---|
| | (mg/tablet) | |
| Active ingredient | 250 | |
| Cellulose, microcrystals | 400 | |
| Silicon dioxide, fumed | 10 | |
| Stearic acid | 5 | |
| Total | 665 | mg |

The components are blended and compressed to form tablets each weighing 665 mg.

### Formulation Example 3

An aerosol solution is prepared containing the following components:

| | Weight |
|---|---|
| Active ingredient | 0.25 |
| Ethanol | 25.75 |
| Propellant 22 (chlorodifluoromethane) | 74.00 |
| Total | 100.00 |

The active compound is mixed with ethanol and the admixture added to a portion of the propellant 22, cooled to −30° C. and transferred to filling device. The required amount is then fed to stainless steel container and diluted with the reminder of the propellant. The valve units are then fitted to the container.

### Formulation Example 4

Tablets, each containing 60 mg of active ingredient, are made as follows.

| | | |
|---|---|---|
| Active ingredient | 60 | mg |
| Starch | 45 | mg |
| Microcrystals cellulose | 35 | mg |
| Polyvinylpyrrolidone | 4 | mg |
| (as 10% solution in | | |
| water) | | |
| Sodium carboxymethyl starch | 4.5 | mg |
| Magnesium stearate | 0.5 | mg |
| Talc | 1 | mg |
| Total | 150 | mg |

The active ingredient, starch, and cellulose are passed through a No. 45 mesh U.S. sieve, and the mixed thoroughly. The aqueous solution containing polyvinylpyrrolidone is mixed with the resultant powder, and the admixture then is passed through a No. 14 mesh U.S. sieve. The granules so produced are dried at 50° C. and passed through a No. 18 mesh U.S. sieve. The sodium carboxymethyl starch, magnesium stearate, and talc, previously passed through No. 60 mesh U.S. sieve, are then added to the granules which, after mixing, are compressed on a tablet machine to yield tablets each weighing 150 mg.

### Formulation Example 6

Capsules, each containing 80 mg of active ingredient, are made as follows:

| | |
|---|---|
| Active ingredient | 80 mg |
| Starch | 59 mg |
| Microcrystals cellulose | 59 mg |
| Magnesium stearate | 2 mg |
| Total | 200 mg |

The active ingredient, starch, cellulose, and magnesium stearate are blended, passed through a No. 45 mesh U.S. sieve, and filled into hard gelatin capsules in 200 mg quantities.

### Formulation Example 6

Suppository, each containing 225 mg of active ingredient, are made as follows:

| | |
|---|---|
| Active ingredient | 225 mg |
| Saturated fatty acid glycerides | 2000 mg |
| Total | 2225 mg |

The active ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

### Formulation Example 7

Suspensions, each containing 50 mg of active ingredient per 5 mL dose, are made as follows:

| Active ingredient | 50 | mg |
|---|---|---|
| Sodium carboxymethyl cellulose | 50 | mg |
| Syrup | 1.25 | mL |
| Benzoic acid solution | 0.10 | mL |
| Flavor | q.v. | |
| Color | q.v. | |
| Purified water to total | 5 | mL |

The active ingredient is passed through a No. 45 U.S. sieve, and mixed with the sodium carboxymethyl cellulose and syrup to form a smooth paste. The benzoic acid solution and flavor are diluted with a portion of the water and added, with stirring. Sufficient water is then added to produce the required volume.

### Formulation Example 8

An intravenous formulation may be prepared as follows:

| | | |
|---|---|---|
| Active ingredient | 100 | mg |
| Saturated fatty acid glycerides | 1000 | mL |

The solution of the above ingredients generally is administered intravenously to a subject at a rate of 1 mL per minute.

### Industrial Applicability

It is provided that type-X sPLA₂ inhibitors are useful in preventing or treating Alzheimer's disease.

1. A composition for prevention or treatment of Alzheimer's disease which contains a type-X sPLA₂ inhibitor as an active ingredient.

2. A composition for prevention or treatment of Alzheimer's disease which contains as an active ingredient a compound represented by the formula (I): 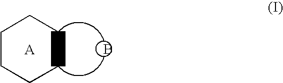
wherein Ring A is represented by the formula (a) to (d): 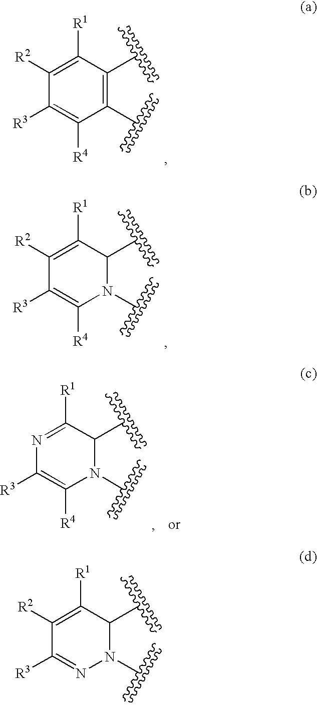
wherein R¹ and R² are each independently hydrogen atom, non-interfering substituent, or —(L¹)-(acidic group) wherein L¹ is an acid linker having an acid linker length of 1 to 5, provided that one of the R¹ and R² is —(L¹)-(acidic group);
R³ and R⁴ are each independently hydrogen atom, non-interfering substituent, carbocyclic group, carbocyclic group substituted with a non-interfering substituent(s), heterocyclic group, or heterocyclic group substituted by a non-interfering substituent(s); and
—B— is represented by the formula (e) to (h): 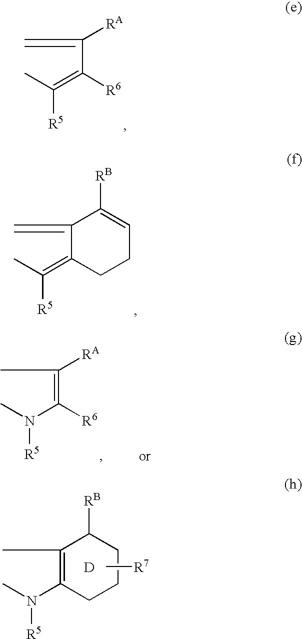
wherein R⁵ is (j) C1 to C20 alkyl, C2 to C20 alkenyl, C2 to C20 alkynyl, carbocyclic group, or heterocyclic group, (k) the group represented by (j) each substituted independently with at least one group selected from non-interfering substituents, or —(L²)—R⁸ wherein L² is a divalent linking group of 1 to 18 atom(s) selected from hydrogen atom(s), nitrogen atom(s), carbon atom(s), oxygen atom(s), and sulfur atom(s), and
R⁸ is a group selected from the groups (j) and (k);
R⁶ is hydrogen atom, halogen, C1 to C3 alkyl, C3 to C4 cycloalkyl, C3 to C4 cycloalkenyl, C1 to C3 alkyloxy, or C1 to C3 alkylthio;
R⁷ is hydrogen atom or non-interfering substituent;
R^{A} is represented by the formula: 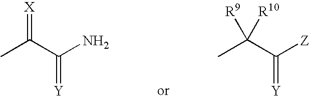
wherein R⁹ and R¹⁰ are each independently hydrogen atom, C1 to C3 alkyl, or halogen;
X and Y are each independently oxygen atom or sulfur atom; and
Z is —NH₂ or —NHNH₂;
R^{B} is —CONH₂ or —CONHNH₂; and,
Ring D is cyclohexene ring or benzene ring;
provided that Ring A is (b), (c), or (d) when —B— is (e) or (f), a prodrug thereof, its pharmaceutically acceptable salt, or its solvate.

3. A composition for prevention or treatment of Alzheimer's disease which contains a compound, a prodrug thereof, its pharmaceutically acceptable salt, or its solvate as claimed in claim 2 as an active ingredient, wherein R¹ is hydrogen atom or —(L³)—R¹¹ wherein L³ is —OCH₂—, —SCH₂—, —NH—CH₂—, —CH₂—CH₂—, —O—CH(CH₃)—, or —O—CH(CH₂CH₂C₆H₅)—; R¹¹ is —COOH, —CONHSO₂C₆H₅, —SO₃H, or —P(O)(OH)₂; and
R² is hydrogen atom or —(L⁴)—R¹² wherein L⁴ is represented by the formula: 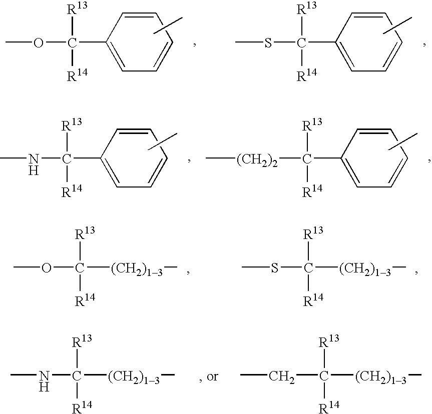
wherein R¹³ and R¹⁴ are each independently hydrogen atom, C1 to C10 alkyl, C1 to C10 aralkyl, carboxy, alkyloxycarbonyl, or halogen; R¹² is —COOH, —SO₃H, or —P(O)(OH)₂, provided R¹ and R² are not hydrogen atom at the same time.

4. A composition for prevention or treatment of Alzheimer's disease which contains a compound, a prodrug thereof, its pharmaceutically acceptable salt, or its solvate as claimed in claim 2 as an active ingredient, wherein R³ is hydrogen atom, C1 to C6 alkyl, C3 to C6 cycloalkyl, aryl, or a heterocyclic group and R⁴ is hydrogen atom or halogen.

5. A composition for prevention or treatment of Alzheimer's disease which contains a compound, a prodrug thereof, its pharmaceutically acceptable salt, or its solvate as claimed in claim 2 as an active ingredient, wherein R⁵ is —(CH₂)₁₋₆—R¹⁵ wherein R¹⁵ is represented by the formula: 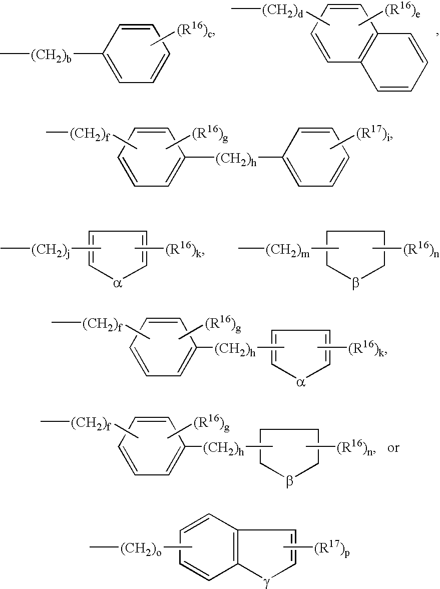
wherein b, d, f, h, j, m, and o are independently an integer from 0 to 2; R¹⁶ and R¹⁷ are each independently halogen, C1 to C10 alkyl, C1 to C10 alkyloxy, C1 to C10 alkylthio, aryloxy, or C1 to C10 haloalkyl; α is oxygen atom or sulfur atom; β is —CH₂— or —(CH₂)₂—; γ is oxygen atom or sulfur atom; c, i, and p are independently an integer from 0 to 5; e is an integer from 0 to 7; g is an integer from 0 to 4; k and n are each independently an integer from 0 to 3.

6. A composition for prevention or treatment of Alzheimer's disease which contains a compound, a prodrug thereof, its pharmaceutically acceptable salt, or its solvate as claimed in claim 5 as an active ingredient, wherein R⁵ is —CH₂—R¹⁸ wherein R¹⁸ is represented by the formula: 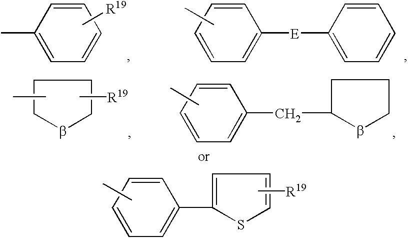
wherein β is —CH₂— or —(CH₂)₂—; R¹⁹ is hydrogen atom, C1 to C3 alkyl, or halogen; E is a bond, —CH₂— or —O—.

7. A composition for prevention or treatment of Alzheimer's disease which contains a compound, a prodrug thereof, its pharmaceutically acceptable salt, or its solvate as claimed in claim 2 as an active ingredient, wherein R¹ is —OCH₂COOH.

8. A composition for prevention or treatment of Alzheimer's disease which contains a compound, a prodrug thereof, its pharmaceutically acceptable salt, or its solvate as claimed in claim 2 as an active ingredient, wherein R² is hydrogen atom.

9. A composition for prevention or treatment of Alzheimer's disease which contains a compound, a prodrug thereof, its pharmaceutically acceptable salt, or its solvate as claimed in claim 2 as an active ingredient, wherein R⁶ is C1 to C3 alkyl.

10. A composition for prevention or treatment of Alzheimer's disease which contains a compound, a prodrug thereof, its pharmaceutically acceptable salt, or its solvate as claimed in claim 2 as an active ingredient, wherein R^{A} is —CH₂CONH₂ or —COCONH₂.

11. A composition for prevention or treatment of Alzheimer's disease which contains a compound as an active ingredient represented by the formula: 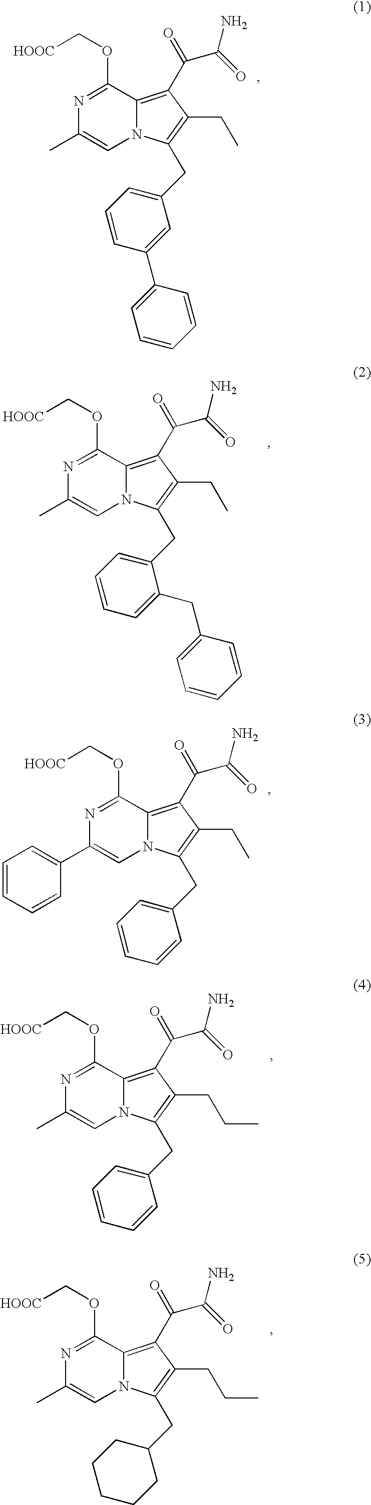 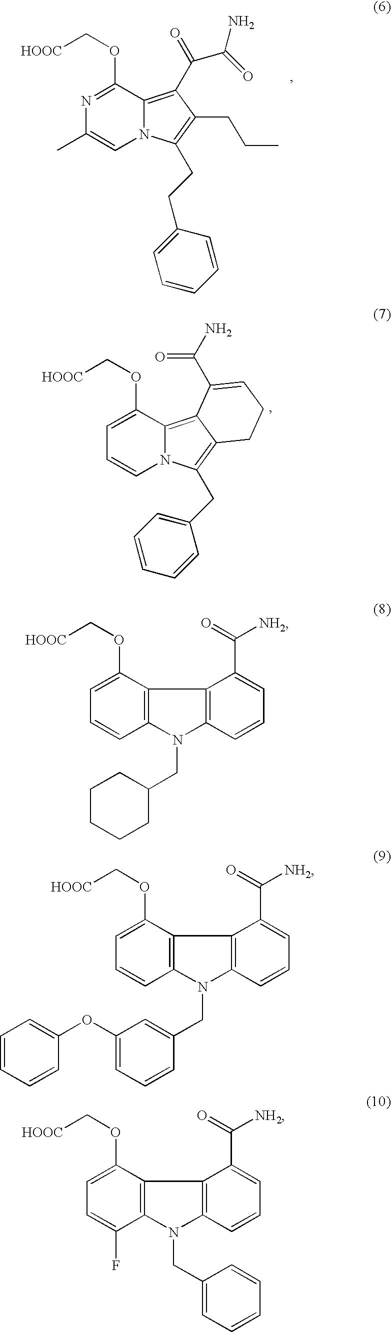 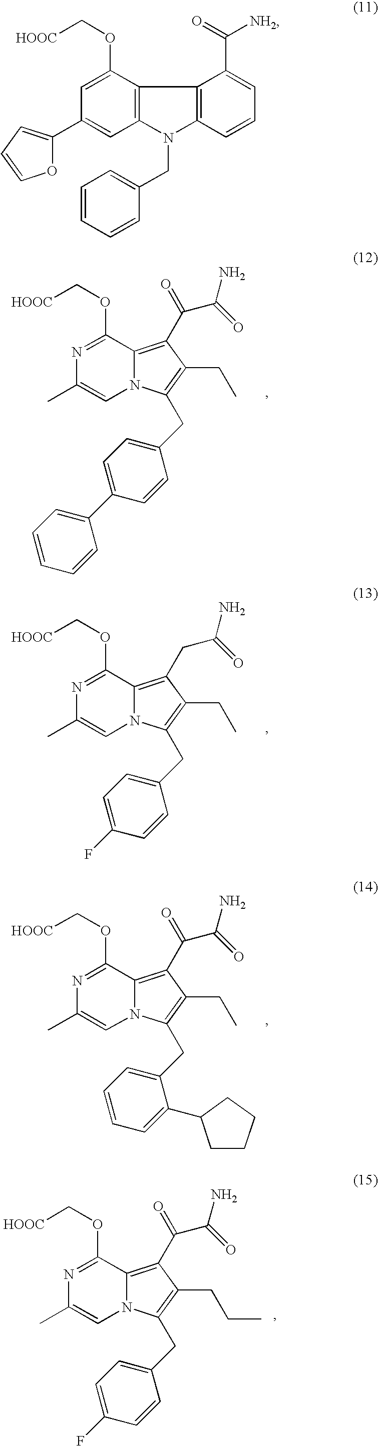 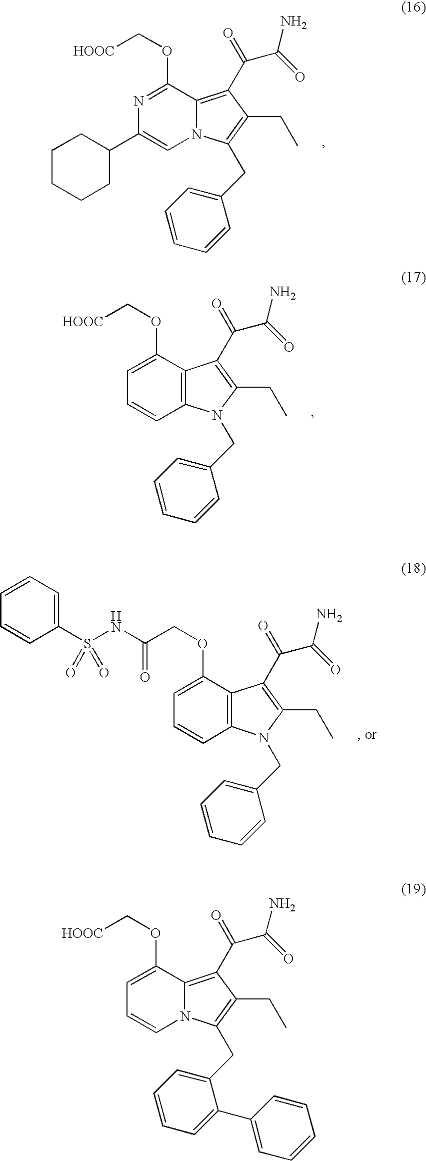
a prodrug thereof, its pharmaceutically acceptable salt, or its solvate.

12. Use of a type-X sPLA₂ inhibitor for the preparation of a medicament for the treatment of Alzheimer's disease.

13. Use as claimed in claim 12 wherein the type-X sPLA₂ inhibitor is the compound claimed in any one of claims 2 to 11.

14. A method of treating a mammal, including a human, to alleviate the pathological effects of Alzheimer's disease, which comprises administration to said mammal of a type-X sPLA₂ inhibitor in a pharmaceutically effective amount.

15. A method as claimed in claim 14 wherein the type-X sPLA₂ inhibitor is the compound claimed in any one of claims 2 to 11.
